(19) United States
(12) Patent Application Publication
(10) Pub. No.: US 2025/0332025 A1
(43) Pub. Date: Oct. 30, 2025

(54) Expandable Thermal Contact Pad

(71) Applicant: C. R. Bard, Inc., Franklin Lakes, NJ (US)

(72) Inventors: Christopher K. Brooks, Lawrenceville, GA (US); Nathaniel Barnes, Covington, GA (US); Cecille A. Canary, Atlanta, GA (US); Younggeun Cho, Covington, GA (US)

(21) Appl. No.: 18/866,907

(22) PCT Filed: May 27, 2022
(86) PCT No.: PCT/US2022/031428

§ 371 Date: Nov. 18, 2024

(51) Int. Cl. A61F 7/02; A61F 7/00

(52) U.S. Cl. CPC A61F 7/02; A61F 2007/0054; A61F 2007/0091

(57) ABSTRACT

Disclosed herein is a medical pad for exchanging thermal energy between a targeted temperature management (TTM) fluid and a patient. The medical pad includes a fluid compartment configured for circulation of a TTM fluid therein. The pad is configured for expansion by providing compressed air to one or more air compartments of the pad, where the expansion defines an increased patient contact area of the pad. An air control module provides air pressure to one or more pads of a TTM system, and the air control module may be incorporated into a TTM system module. The pad further includes a hydrogel layer disposed across an under side of the pad.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems include a TTM fluid control module coupled to at least one contact pad via a fluid deliver line. One such system including a thermal contact pad is disclosed in U.S. Pat. No. 11,234,859 titled “Medical Pad and System for Thermotherpy” filed Oct. 9, 2019, which is incorporated herein by reference in its entirety.

Patients have different shapes and sizes. Providing a reasonable number of thermal contact pads having different sizes and shapes limits the effectivity of the TTM therapy for many patients. Systems and thermal contacts disclosed herein address the forgoing.

### SUMMARY OF THE INVENTION

Briefly summarized, disclosed herein is a medical pad for exchanging thermal energy between a targeted temperature management (TTM) fluid and a patient. According to some embodiments, the medical pad includes a fluid compartment configured for circulation of the TTM fluid therein, the fluid compartment defining a patient contact area of the pad. The medical pad further includes an air compartment coupled with the fluid compartment, the air compartment configured to define a lateral expansion of the fluid compartment based on an air pressure within the air compartment.

In some embodiments, the fluid compartment is configured to expand in accordance with an increase in the air pressure.

In some embodiments, the fluid compartment is configured to expand along a length dimension in accordance with an increase of the air pressure.

In some embodiments, the fluid compartment is configured to expand along a width dimension in accordance with an increase of the air pressure.

In some embodiments, the fluid compartment is configured to contract in accordance with a decrease of the air pressure.

In some embodiments, the air compartment is coupled with the fluid compartment along one or more perimeter edges of the fluid compartment.

In some embodiments, the air compartment is coupled with the fluid compartment along a first permitter edge and along a second perimeter edge, the second perimeter edge disposed opposite the first perimeter edge.

In some embodiments, the fluid compartment is configured for circulation of the TTM fluid therein when the TTM fluid defines a negative pressure, in some embodiments, the fluid compartment includes a support structure configured to define a minimum thickness of the fluid compartment. In some embodiments, the air compartment is constructed to prevent expansion of the pad when the air pressure therein is positive.

In some embodiments, the air compartment defines a number of tubular segments in fluid communication with each other, where the tubular segments are configured to lengthen based on the air pressure therein, and where the tubular segments are configured to prevent diametral expansion based on the air pressure therein.

In some embodiments, one or more tubular segments includes a bellows defining a bias toward a non-expanded state.

In some embodiments, one or more tubular segments include a number of expansion joints. In some embodiments, one or more of the number expansion joints are configured to (i) maintain a non-expanded state of the one or more tubular segments when the air pressure within the air compartment is below a first defined expansion pressure and (ii) define an expanded state of the one or more tubular segments when the air pressure within the air compartment exceeds the first defined expansion pressure.

In some embodiments, the fluid compartment is formed of a stretchable material to enable the fluid compartment to stretch between a first contact area and a second contact area, the second contact area greater than the first contact area.

In some embodiments, the fluid compartment includes one or more folds extending across the fluid compartment. In some embodiments, the one or more of the number folds are configured to (i) maintain a folded state defining the first contact area when the air pressure within the air compartment is below the first defined expansion pressure and (ii) become unfolded defining the second contact area when the air pressure within the air compartment exceeds the first defined expansion pressure.

In some embodiments, the fluid compartment includes a hydrogel layer disposed across an underside thereof.

Also disclosed herein is a system for providing a targeted temperature management (TTM) therapy to a patient. The system includes: (i) a thermal contact pad according to any of the medical pad embodiments described above; (ii) a TTM control module coupled with the thermal contact pad via a fluid delivery line, where the TTM control module is configured to circulate the TTM fluid within the fluid compartment at a defined temperature in accordance with the TTM therapy; and (iii) an air control module coupled with the thermal contact pad via an air delivery line, where the air control module is configured to define the air pressure within the air compartment of the thermal contact pad.

In some embodiments of the system, the air control module is configured to define the air pressure in accordance with an input from a clinician.

In some embodiments of the system, the air control module is configured to prevent the air pressure from exceeding a predefined maximum pressure limit.

In some embodiments of the system, the air control module is integrally incorporated within the TTM control module.

Also disclosed herein is a method of providing a targeted temperature management (TTM) therapy to a patient. The method includes: (i) applying a thermal contact pad to the patient; (ii) circulating a TTM fluid at a defined temperature through a fluid compartment of the pad to define a thermal energy exchange between the TTM fluid and the patient, and (iii) providing an air pressure to an air compartment of the thermal contact pad to increase a patient contact area of the thermal contact pad, thereby enhancing the thermal energy exchange.

In some embodiments of the method, the increase of the patient contact area includes a lateral expansion of the fluid compartment in a first direction. In some embodiments of the method, the increase of the patient contact area further includes an expansion of the fluid compartment in a second direction orthogonal to the first direction.

In some embodiments of the method, increasing of the patient contact area includes unfolding one or more folds extending across the fluid compartment. In some embodiments of the method, increasing of the patient contact area includes lengthening one or more tubular segments of the air compartment.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a targeted temperature management (TTM) system including a thermal contact pad coupled with a patient, in accordance with some embodiments;

FIG. 1B illustrates a top view of an expandable thermal contact pad of the system of FIG. 1A, in accordance with some embodiments;

FIG. 2A illustrates an exemplary embodiment of an expandable thermal contact pad, in accordance with some embodiments;

FIG. 2B is a cross-sectional side view of the expandable thermal contact pad of FIG. 2A, in accordance with some embodiments;

FIG. 2C illustrates a cross-sectional side view of another embodiment of the thermal contact pad of FIG. 2A, in accordance with some embodiments;

FIG. 3A illustrates a portion of an air compartment of the thermal contact pad of FIG. 2A having a bellows, in accordance with some embodiments; and

FIG. 3B illustrates a portion of an air compartment of the thermal contact pad of FIG. 2A having expansion joints, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, fluid, and thermal interaction. Two components may be connected to or coupled with each other even though they are not in direct contact with each other. For example, two components may be coupled with each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

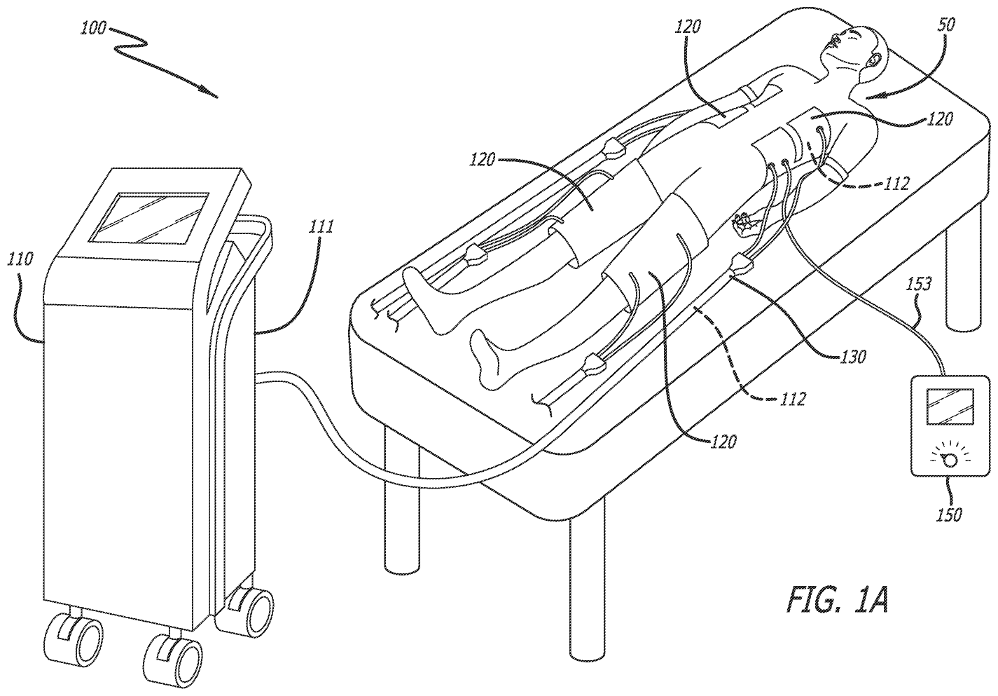

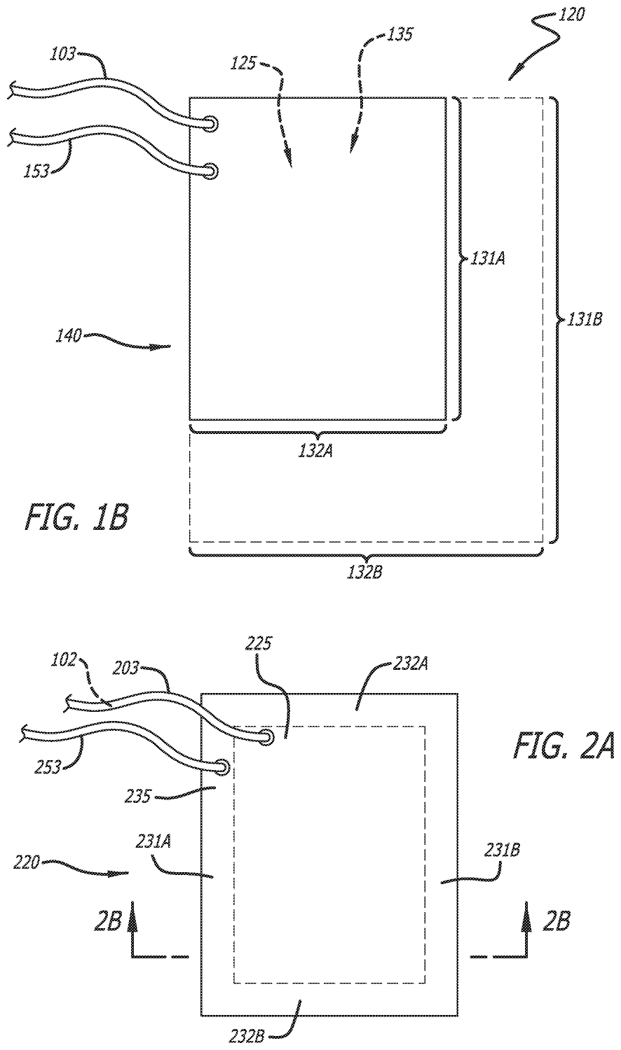

FIG. 1A illustrates a targeted temperature management (TTM) system 100 connected to a patient 50 for administering TTM therapy to the patient 50 which may include a cooling and/or warming of the patient 50, in accordance with some embodiments. The TTM system 100 includes a TTM module 110 for preparing and delivering TTM fluid 102. The TTM system 100 includes a fluid deliver line (FDL) 103 extending from the TTM module 110 to a thermal contact pad (pad) 120 to provide for flow of TTM fluid 102 between the TTM module 110 and the pad 120. In some embodiments, the TTM module 110 may deliver TTM fluid 102 to the pad 120 so that the pressure of the TTM fluid 102 is negative (i.e., less than an environmental pressure).

The TTM system 100 may include 1, 2, 3, 4 or more pads 120 and the TTM system 100 may include 1, 2, 3, 4 or more fluid delivery lines 103 in accordance with the number of pads 120. In use, the TTM module 110 prepares the TTM fluid 102 for delivery to the pad 120 by heating or cooling the TTM fluid 102 to a defined temperature in accordance with a prescribed TTM therapy. The TTM module 110 circulates the TTM fluid 102 within a fluid compartment 125 of the pad 120 by way of the fluid delivery line 103 to facilitate thermal energy exchange with the patient 50. During the TTM therapy, the TTM module 110 may continually control the temperature of the TTM fluid 102 toward a target TTM temperature. As shown, the pad 120 may be applied to different body parts of the patient 50. As such, the pad 120 may be available in different configurations, such as sizes and shapes, for example, to accommodate the different body parts.

The system 100 further includes an air control module 150. The air control module 150 may be an air compressor or any other type of air source, such as an air tank or a facility air source, for example. In some embodiments, the air source 150 may be integral to the TTM module 110.

The air control module 150 is coupled with the pad 120 via an air delivery line 153. As the system may include multiple pads 120, the air control module 150 may be coupled with multiple pads 120 via multiple air delivery lines 153.

All or a subset of the pads 120 may be configured for expansion, where expansion of the pad 120 defines an increase in the patient contact area of the pad 120. A magnitude of thermal exchange with the patient via the pad 120 is at least partially defined the patient contact area of the pad 120. In some instances, it may be advantageous during a TTM therapy to maximize the patient contact area. A total patient contact area may be defined by a sum of the individual patient contact areas of the pads 120. A pad 120 configured for expansion may facilitate a desired contact area for different patient sizes and shapes. For example, the pad 120 in a non-expanded state may define a less than desired patient contact area for a patient. Similarly, the pad 120 in an expanded state may provide for the desired contact area for the patient. The pad 120 is configured for expansion in accordance with the air pressure supplied to the pad 120 via the air control module 150.

The air control module 150 may be configured to provide an air pressure to the pad 120 according to different modes of operation. For example, in some embodiments, the air control module 150 may be configured to supply air pressure at a single non-adjustable magnitude (i.e., pressure level). In other embodiments, the air control module 150 may be configured to supply a variably magnitude of air pressure across a predefined magnitude range. In still other embodiments, the air control module 150 may be configured to supply air pressure at a number of predefined magnitudes. In some embodiments, the air control module 150 may be configured to supply air pressure to the pad 120 in accordance with an input from the clinician. In some embodiments, the air control module 150 may be configured to limit the air pressure to the pad 120 so as to be below a predefined maximum pressure limit.

As the system may include multiple pads 120, the air control module 150 may include a number of channels in accordance with the multiple pads 120. As such, the air control module 150 may provide air pressure to each of the multiple pads 120 at the same or different magnitudes.

FIG. 1B illustrates a top side view of the pad 120. The pad 120 is coupled with the air delivery line 153 to enable coupling with the air control module 150. The pad 120 is configured to expand in accordance with an air pressure supplied by the air control module 150 to an air compartment 135 of the pad 120. In the illustrated embodiment, pad 120 defines a rectangular shape. However, in other embodiments, the pad 120 may define other shapes, such as a circle, an oval, for example. In some embodiments, the pad 120 may be shaped to accommodate different portions of the patient body, such as the legs, arms, neck, hips, or torso, for example. The pad 120 includes fluid compartment 125 and one or more air compartments 135, where the fluid compartment 125 is coupled with the fluid delivery line, and where the air compartment is coupled with the air delivery line 153.

The pad 120 may generally define a first length 131A when a first air pressure (P1) is supplied to the pad 120. Similarly, the pad 120 may generally define a first width 132A when P1 is supplied to the pad 120. The pad 120 may also define a second length 131B and/or a second width 132B when a second pressure P2 is supplied to the pad 120, where (i) the second length 131B is greater than the first length 131A, (ii) the second width 132B is greater than the first width 132A, and (iii) P2 is greater than P1. In some instances, P1 may be a zero “0” gauge pressure and P2 may be a positive pressure. In some embodiments, the pad 120 may be configured to expand in only one direction, such as along the width or the length. In other embodiments, the pad 120 may be configured to expand in two directions, such as along the width and the length. In some embodiments, the pad 120 may include a hydrogel layer 140 disposed across an underside of the pad 120.

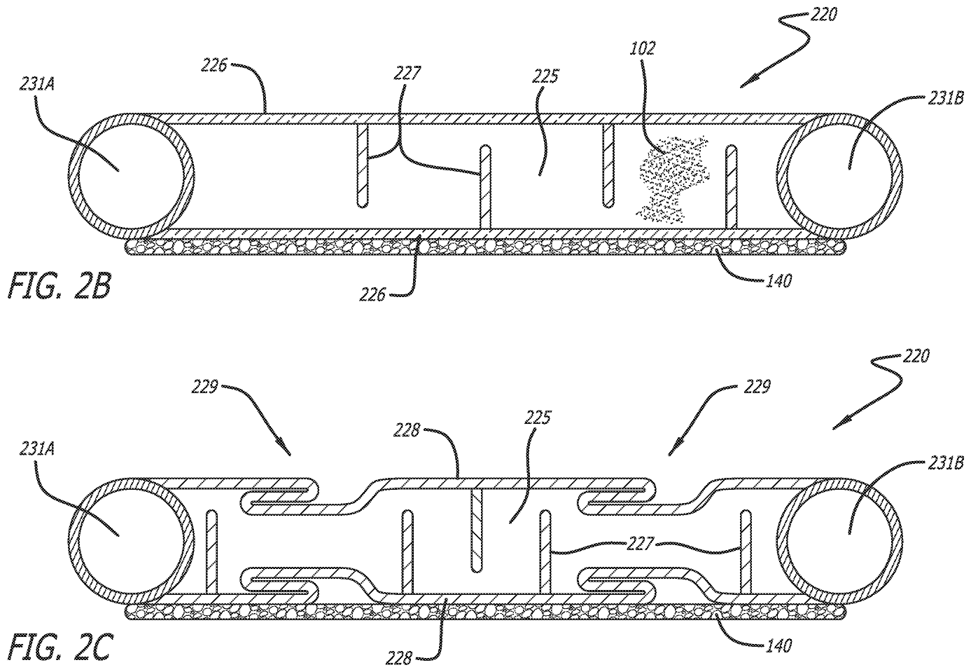

FIGS. 2A-2B illustrate an exemplary embodiment of a pad 220 which may define the features and functionality of the pad 120 described above. FIG. 2A is a top view of the pad 220 and FIG. 2B is a cross-sectional illustration of the pad 220 cut along sectioning lines 2B-2B. The pad 220 includes a fluid compartment 225 surrounded by an air compartment 235. The fluid compartment 225 is coupled with a fluid delivery line 203 which supplies a TTM fluid 102 (see FIG. 1A) to the fluid compartment 225. Similarly, the compartment 235 is coupled with an air delivery line 253 which supplies an air pressure to the air compartment 235.

The air compartment 235 may be formed of sub-compartments 231A, 231B, 232A, and 232B. The sub-compartments 231A, 231B extend across a length of the pad 220, and are along opposite sides of the pad 220. The sub-compartments, 231A, 231B may generally define the length of the fluid compartment 225. Similarly, the sub-compartments 232A, 232B extend across a width of the pad 220, and are disposed along opposite sides of the pad 220, and the sub-compartments 232A, 232B may generally the define the width of the fluid compartment 225.

Each sub-compartment may define a tubular cross section. The structure of each sub-compartment may be configured such that radial expansion of the tubular cross section of the sub-compartment is limited or prevented when air pressure is supplied to the sub-compartment. Conversely, each sub-compartment is configured for longitudinal expansion of the sub-compartment when the air pressure is supplied to the sub-compartment. More specifically, the sub-compartments 231A, 231B may increase in length in response to the air pressure supplied to the air compartment 235 to cause the length of the pad 220 to increase. Similarly, the sub-compartments 232A, 232B may increase in length in response to the air pressure supplied to the air compartment 235 to cause the width of the pad 220 to increase.

The fluid compartment 225 may be formed of a sheet material 226 that is expandable in the length and/or the width directions. In some embodiments, the sheet material 226 may be generally stretchable, such as an elastomeric material, for example.

With further reference to FIG. 2B, the fluid compartment 225 may include a support structure 227 to maintain a minimum thickness of the fluid compartment 225. The support structure 227 may include a number of columns or walls that extended between a top side and a bottom side of the fluid compartment 225. The support structure 227 may be configured to prevent a collapse of the fluid compartment 225 in the case of an external crushing force applied thereto or in the case of a negative fluid pressure of the TTM fluid 102 within the fluid compartment 225.

FIG. 2C illustrates another embodiment of the pad 220, where the fluid compartment 225 is formed of a non-stretchable material 228. In this embodiment, the fluid compartment 225 includes a number of folds 229 defining expansion joints extending across the fluid compartment 225 in the width direction. Although not shown, the fluid compartment 225 may also include a number of folds 229 defining expansion joints extending across the fluid compartment 225 in the length direction. The fluid compartment 225 defines the non-expanded state when the folds 229 are folded and the fluid compartment 225 defines the expanded state when the folds 229 are unfolded. The folds 229 may be configured to remain folded when a pressure equal to or less than P1 is supplied to the air compartment 135 and become unfolded when P2 is supplied to the air compartment 135, where P2 is greater than P1. Similar to the embodiment of FIG. 2B, the embodiment of FIG. 2C may include the support structure 227 configured to prevent a collapse of the fluid compartment 225 in the case of an external crushing force applied thereto or in the case of a negative fluid pressure of the TTM fluid 102 within the fluid compartment 225.

In some embodiments, the pad 220 may be configured for expansion in only a single direction, such as across the length or the width. In such embodiments, the sub-compartments 231A, 231B or the sub-compartments 232A, 232B may be omitted.

In some embodiments, the pad 220 may be configured for independent expansion along the length and the width. In other words, the sub-compartments 231A, 231B may be coupled with one channel of the air control module 150 via one air delivery line 153 and the sub-compartments 232A, 232B may be coupled with a different channel of the air control module 150 via a different air delivery line 153. As such, the pad 220 may be independently expanded along the length direction by supplying air pressure to the sub-compartments 231A, 231B and may also be independently expanded along the width direction by supplying air pressure to the sub-compartments 232A, 232B.

The pad 220 is just one embodiment of an expandable thermal contact pad. It appreciated that one of ordinary skill may contemplate other structural arrangements of a fluid compartment combined with one or more air compartments to define the expanding functionality of the pad 120, which other structural arrangements are disposed herein. It is noted also that the embodiment of FIGS. 2A-2B and the embodiment of FIG. 2C are not mutually exclusive, i.e., the sheet material 226 may include one or more folds 229 and the sheet material 228 may be stretchable or include stretchable portions.

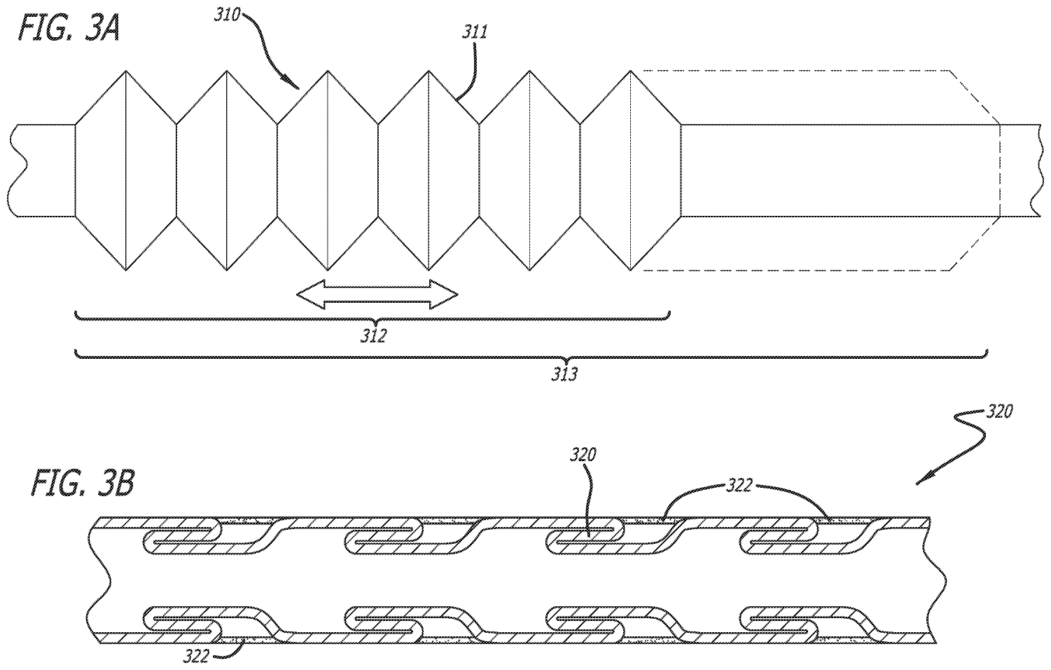

FIG. 3A illustrates a portion of a sub-compartment 310 that can be incorporated into any one of the sub-compartments 231A, 231B, 232A, or 232B of FIGS. 2A-2C. The sub-compartment 310 is generally configured to lengthen in accordance with a variable air pressure supplied thereto. In other words, the sub-compartment 310 is configured for continuous expansion in accordance with an air pressure that is continuously variable. The sub-compartment 310 includes a bellows 311 that is biased toward a non-expanded length 312, which may be defined when a zero (0) pressure is supplied to the sub-compartment 310. The bellows 311 transitions to an expanded length 313 when an increased magnitude of air pressure is supplied to the sub-compartment 310 to define an expanded state of the pad 120 (FIGS. 1A-1B). The bellows 311 also transitions back to the non-expanded length 312 when the air pressure is removed to the sub-compartment 310 to define the non-expanded state of the pad 120.

FIG. 3B illustrates a portion of a sub-compartment 320 that can be incorporated into any one of the sub-compartments 231A, 231B, 232A, or 232B of FIGS. 2A-2C. The sub-compartment 320 is generally configured to lengthen in accordance with an air pressure supplied thereto. The sub-compartment 310 includes a number of expansion joints 321 (e.g., annular folds). Each expansion joint 321 is configured to remain folded a pressure below P1 is supplied to the sub-compartment 320 and become unfolded when an increased pressure (i.e., a pressure greater than P1) is supplied to the sub-compartment 320 thereby increasing a length of the sub-compartment 320. Each expansion joint 321 may include a frangible member 322 configured to (i) maintain the expansion joint 321 in the folded configuration when the pressure within the sub-compartment 320 is less than P1 and (ii) release the expansion joint 321 from the folded configuration when the pressure is greater than P1.

In some embodiments, one subset of the expansion joints 321 may be configured to unfold when the pressure exceeds P1 thereby defining a first expanded length. Another subset of expansion joints 321 may be configured to unfold when the pressure exceeds P2, where P2 is greater than P1, thereby defining a second expanded length greater than the first expanded length.

It is noted also that the embodiment of FIGS. 3A and the embodiment of FIG. 3B are not mutually exclusive, i.e., all or a subset of the sub-compartments 231A, 231B, 232A, or 232B may include a bellows 311 and a number of expansion joints 321.

Providing a TTM therapy to a patient may include all or a subset of the flowing steps or process. One step may include applying the thermal contact pad to the patient. Another step may include circulating a TTM fluid at a defined temperature through a fluid compartment of the pad to define a thermal energy exchange between the TTM fluid and the patient. Another step may include providing an air pressure to an air compartment of the thermal contact pad to increase a patient contact area of the thermal contact pad to enhance the thermal energy exchange.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical pad for exchanging thermal energy between a targeted temperature management (TTM) fluid and a patient, the pad comprising:
a fluid compartment configured for circulation of the TTM fluid therein, the fluid compartment defining a patient contact area of the pad; and
an air compartment coupled with the fluid compartment, the air compartment configured to define a lateral expansion of the fluid compartment based on an air pressure within the air compartment.

2. The pad of claim 1, wherein the fluid compartment is configured to expand in accordance with an increase of the air pressure.

3. The pad of claim 1, wherein the fluid compartment is configured to expand along a length dimension in accordance with the increase of the air pressure.

4. The pad of claim 1, wherein the fluid compartment is configured to expand along a width in accordance with the increase of the air pressure.

5. The pad of claim 1, wherein the fluid compartment is configured to contract in accordance with a decrease of the air pressure.

6. The pad of claim 1, wherein the air compartment is coupled with the fluid compartment along one or more perimeter edges of the fluid compartment.

7. The pad of claim 6, wherein the air compartment is coupled with the fluid compartment along a first permitter edge and along a second perimeter edge, the second perimeter edge disposed opposite the first perimeter edge.

8. The pad of claim 1, wherein the fluid compartment includes a support structure configured to define a minimum thickness of the fluid compartment.

9. The pad of claim 1, wherein the fluid compartment is configured for circulation of the TTM fluid therein when the TTM fluid defines a negative pressure within the fluid compartment.

10. The pad of claim 1, wherein the air compartment is constructed to prevent expansion of the pad in a thickness direction when the air pressure within the air compartment is positive.

11. The pad of claim 10, wherein:
the air compartment defines a number of tubular segments in fluid communication with each other,
the tubular segments are configured to lengthen based on the air pressure therein, and
the tubular segments are configured to prevent diametral expansion in response to the air pressure therein.

12. The pad of claim 11, wherein:
one or more of the number of tubular segments includes a bellows configured to expand lengthwise based on the air pressure therein, and
the bellows is biased toward a non-expanded state.

13. The pad of claim 10, wherein one or more of the number of tubular segments includes a number of expansion joints.

14. The pad of claim 13, wherein one or more of the number expansion joints are configured to:
maintain a non-expanded state when the air pressure within the air compartment is below a defined expansion pressure, and
define an expanded state when the air pressure within the air compartment exceeds the defined expansion pressure.

15. The pad of claim 1, wherein the fluid compartment is formed of a stretchable material to enable the fluid compartment to stretch between a first contact area and a second contact area, the second contact area greater than the first contact area.

16. The pad of claim 1, wherein the fluid compartment includes one or more folds extending across the fluid compartment.

17. The pad of claim 16, wherein one or more of the number folds are configured to:
maintain a folded state defining the first contact area when the air pressure within the air compartment is below the defined expansion pressure, and
become unfolded defining the second contact area when the air pressure within the air compartment exceeds the defined expansion pressure.

18. The pad of claim 1, wherein the fluid compartment includes a hydrogel layer disposed across an underside of the fluid compartment.

19. A system for providing a targeted temperature management (TTM) therapy to a patient, comprising:
a medical pad according to claim 1;
a TTM control module coupled with the thermal contact pad via a fluid delivery line, the TTM control module configured to circulate a TTM fluid within the fluid compartment at a defined temperature in accordance with the TTM therapy; and
an air control module coupled with the thermal contact pad via an air delivery line, the air control module configured to define the air pressure within the air compartment of the thermal contact pad.

20. The system of claim 19, wherein the air control module is configured to define the air pressure in accordance with an input from a clinician.

21. The system of claim 19, wherein the air control module is configured to prevent the air pressure from exceeding a predefined maximum pressure limit.

22. The system of claim 19, wherein the air control module is integrally incorporated within the TTM control module.

23-27. (canceled)
